(19) United States
(12) Patent Application Publication
(10) Pub. No.: US 2012/0095482 A1
(43) Pub. Date: Apr. 19, 2012

(54) ENDOPROSTHETIC TEXTILE SCAFFOLD

(76) Inventors: Dale Peterson, La Jolla, CA (US); Ralph Mattern, San Diego, CA (US); Peter Popper, Wilmington, DE (US); Richard Emmitt, Oldwick, NJ (US); Said Rizk, Salem, NH (US); Kevin Ohashi, Jamaica Plain, MA (US); Robert J. Ball, West Olive, MI (US)

(21) Appl. No.: 13/105,728

(22) Filed: May 11, 2011

Related U.S. Application Data
(60) Provisional application No. 61/333,357, filed on May 11, 2010.
(60) Provisional application No. 61/424,531, filed on Dec. 17, 2010.

(51) Int. Cl. A61B 17/03

(52) U.S. Cl. 606/151

(57) ABSTRACT

The endoprosthetic textile scaffold (1) according to an embodiment of the invention includes a first weave (10) substantially planar, including a warp (11) oriented in a first direction (D10) and a weft (13) oriented perpendicularly to the first direction, and a second weave (20) substantially planar, including a warp (21) oriented in a second direction (D20) and a weft (23) oriented perpendicularly to the second direction. The second weave is arranged and bound to the first weave so that the first and second weaves are superimposed in a parallel manner, with the first direction being non-parallel to the second direction.

### CROSS-REFERENCE TO RELATED APPLICATIONS

This application claims the benefit of U.S. Provisional Patent Application Ser. No. 61/333,357, filed on May 11, 2010, and of U.S. Provisional Patent Application Ser. No. 61/424,531, filed on Dec. 17, 2010, both of which are incorporated by reference herein in their entireties for all purposes.

### TECHNICAL FIELD

Embodiments of the present invention relate to an endoprosthetic textile scaffold.

### BACKGROUND

Existing endoprostheses may be made from human or animal tissues. However, these biologic grafts have poor mechanical properties, which can induce their recurrent tear in use.

Synthetic scaffolds may also be used for an endoprosthesis. For example, existing scaffolds are often complex to manufacture and have limited mechanical properties. Scaffolds which are made by embroidery equipment can be mechanically efficient, but must be implanted carefully because their mechanical properties are anisotropic, and they also have predetermined shapes.

### SUMMARY

Embodiments of the present invention may be used in particular, but not exclusively, to repair human tendons and ligaments, in particular the rotator cuff tissue in a human shoulder. Surgical repair of the rotator cuff tissue is usually accomplished by reattaching the tendons or the ligaments in apposition to the region of bone from which they tore. Such a surgical repair is performed as an open procedure or a fully arthroscopic procedure.

However, when direct reattachment is not possible, especially because of a large defect of the bone, the defect is filled by interposing an endoprosthesis. Such an endoprosthesis is also used to improve the strength of the repair.

Embodiments of the present invention include an endoprosthetic textile scaffold which combines great mechanical and healing properties and an ease of use.

One of the ideas underlying an embodiment of the invention is a bi-layer woven scaffold with its layers bound off-axis to each other. In this way, the mechanical properties of the scaffold are substantially isotropic, which enables surgeon to implant the scaffold in any desired orientation with respect to a tendon or ligament to be repaired. Besides, the woven structure of the scaffold may be adapted to include, for example by impregnation, a delivery of biologic agents, in particular those biologic agents which encourage healing. Under these circumstances, the scaffold according to an embodiment of the invention may be used to be sandwiched between a soft tissue, as a tendon or a ligament, and a bone to encourage healing of the interface between these two human tissues. For example, the scaffold may be used to repair the rotator cuff tissue in a human shoulder. The scaffold can also be sandwiched between two soft tissues or two bone interfaces to facilitate healing, according to embodiments of the present invention.

The novel structures described below produce scaffolds which can be trimmed to shape without unraveling, maintain high porosity under tensile and/or compressive loads, and have unique mechanical properties that promote the formation of new tissue interfaces. For example, when placed between bone and tendon the scaffold stabilizes the initial callus yet transmits enough tensile load to produce a transition from bone to tendon tissue within the scaffold as the callus remodels. A textile structure that encourages bone ingrowth is novel, but one which can also encourage tendon ingrowth is extremely valuable. Clinicians have been searching for a method to generate new bone/tendon interface for decades.

According to some embodiments of the present invention, the at least two weaves of the scaffold are each a leno weave. Such a leno weave has the advantage of being cutable in arbitrary shapes, without unraveling or fraying. A leno weave may be easy to manufacture and arrange in the scaffold according to embodiments of the invention. In some embodiments of the present invention, the at least two weaves are made of fibers, especially monofilament fibers, which improve the human tissue growth into the scaffold. Furthermore, such fibers may be made of TephaFlex, which allows making resorbable monofilaments woven as a leno weave.

In addition, according to some embodiments of the invention, the at least two weaves of the scaffold are coated, especially to form a porous sponge, in particular a porous sponge made of collagen, fibrin, or other proteins, proteoglycans, polysaccharides, or polymers.

In practice, the scaffold according to an embodiment of the invention can be implanted using an open procedure or using minimal invasive arthroscopic surgical techniques.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic exploded view of a scaffold according to an embodiment of the present invention;

FIG. 2 is a schematic elevation taken along II of FIG. 1;

FIG. 3 is a view similar to FIG. 2, showing only one of the weaves shown in FIGS. 1 and 2;

FIGS. 4 and 5 are views similar to FIG. 3, respectively showing variants of the aforesaid weave;

FIG. 6 is a view similar to FIG. 2, showing a second embodiment of the present invention;

FIG. 7 illustrates a histological photograph of bone ingrowth; and

FIG. 8 illustrates another histological photograph of bone ingrowth.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

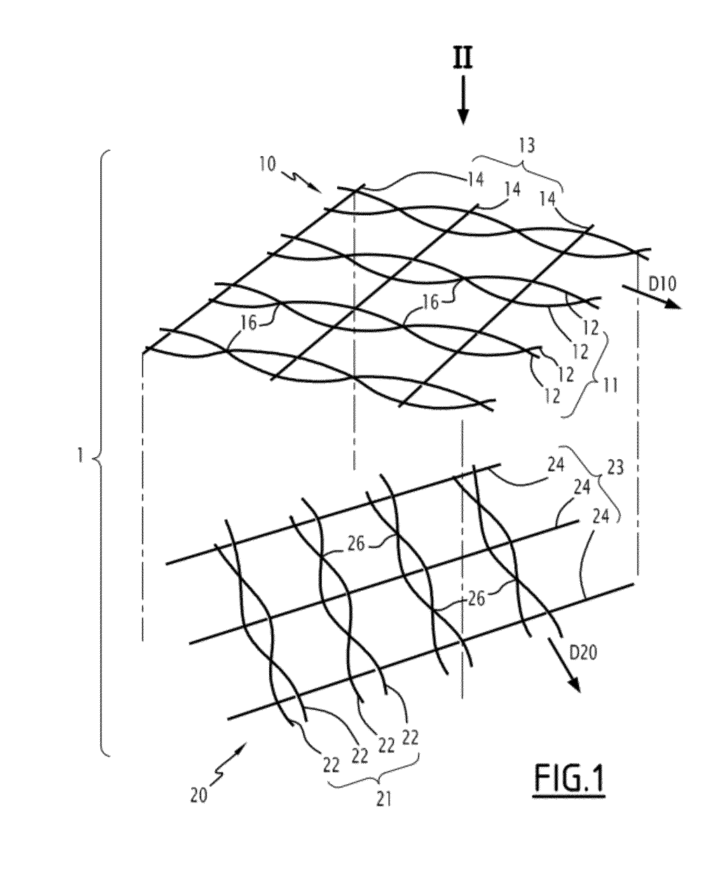

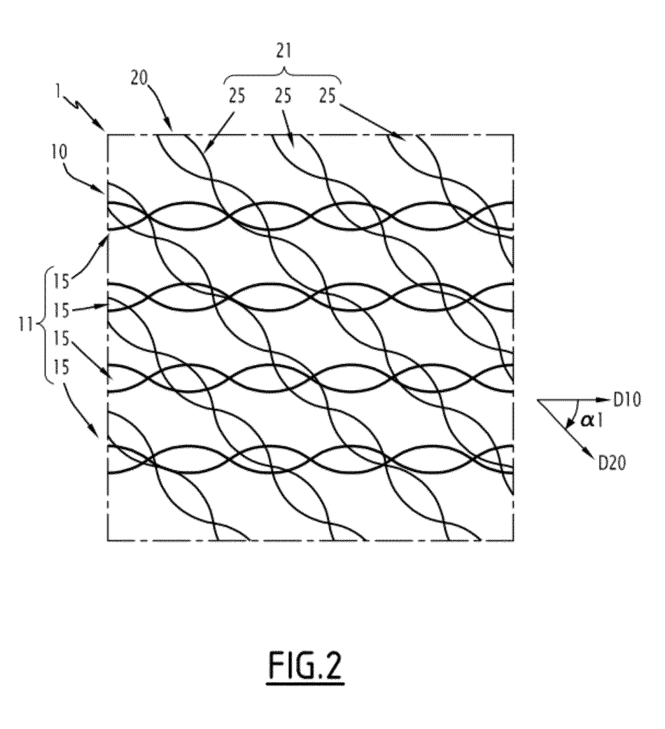

FIGS. 1 and 2 depict an endoprosthetic textile scaffold 1 comprising a first weave 10 and a second weave 20. In the shown embodiment, the two weaves 10 and 20 are identical. More precisely, these two weaves 10 and 20 are substantially planar, that is to say that they correspond each to a bi-dimensional woven textile. Each weave 10, 20 includes a warp 11, 21 consisting of warp fibers 12, 22 oriented in length in a respective direction referenced D10, D20. Each weave 10, 20 further includes a weft 13, 23 consisting of weft fibers 14, 24.

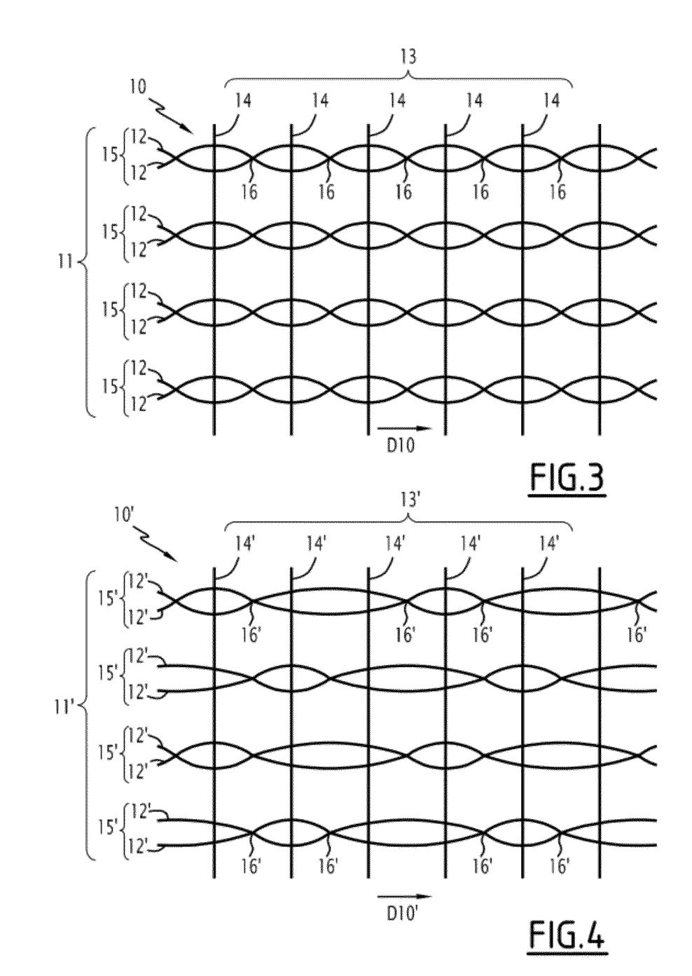

As better shown in FIG. 3 for weave 10, the weft fibers 14 are parallel to each other and run perpendicularly to direction D10, so that they interlace the warp fibers 12. In the same way, in weave 20, the weft fibers 24 are parallel to each other and run perpendicularly to direction D20, so that they interlace the warp fibers 22.

As best shown in FIG. 3 for weave 10, the warp fibers 12 of weave 10, respectively and the warp fibers 22 of weave 20 are not parallel to each other but are associated by pairs 15, 25 of two warp fibers twisting around each other. The two warp fibers 12, 22 of each of these pairs 15, 25 cross at intersections 16, 26. These intersections 16, 26 are successive along the two warp fibers of each pair 15, 25, that is to say along direction D10, D20. As such, each of these intersections 16, 26 between the two warp fibers of each of these pairs 15, 25 is located between two adjacent weft fibers 14, 24. In this way, the twisted warp fibers 12, 22 of each pair 15, 25 are locked in place with respect to weft 13, 23. In other words, the twisted warp fibers 12, 22 of each pair 15, 25 are prevented from slipping along the weft fibers 14, 24. As such, each weave 10, 20 does not unravel easily.

In practice, the textile woven structure, as described above, corresponds to a leno structure according to embodiments of the present invention. In other words, each weave 10, 20 may be a leno weave. As explained above, these leno weaves 10, 20 can be cut in arbitrary shapes without unraveling.

According to a non limiting example, the diameter of the fibers 12, 14, 22 and 24 is about 50 μm or about 150 μm, more than fifteen warp pairs 15, 25 are provided per inch, and more than twenty weft fibers 14, 24 are provided per inch.

As schematically shown in FIGS. 1 and 2, the two weaves 10 and 20 are specifically positioned to each other in scaffold 1. More precisely, these two weaves 10 and 20 are arranged to be parallel superimposed. In other words, the respective planes, in which weaves 10 and 20 are lying respectively, are positioned parallel to each other. In this parallel configuration, the two directions D10 and D20 are positioned to be non parallel to each other. In other words, as shown in FIG. 2, in which weft 13, 23 of each weave 10, 20 is not depicted for clarity of the figure, the two directions D10 and D20 form between them an angle α1 that is different from 0° and 180°. It can be noted that, according to strict geometrical consideration, the aforesaid angle al is defined by the projections of directions D10 and D20 in a plane parallel to weaves 10 and 20.

As shown in FIG. 2, the warp fibers 12 of weave 10 run on the warp fibers 22 of weave 20, being biased with respect to the warp fibers 22 under angle α1, according to embodiments of the present invention. Of course, the same angulation may be found between the weft fibers 14 of weave 10 and the weft fibers 24 of weave 20.

By that way, the mechanical properties of scaffold 1 are not anisotropic, especially in a main direction, but, on the contrary, these mechanical properties tend to be anisotropic, due at least in part to the respective mechanical effects provided by weave 10 and by weave 20. According to some embodiments of the present invention, angle α1 is about 45°. The aforesaid value of 45° includes the same value in either a clockwise direction or in a counterclockwise direction: in an angularly oriented plane, the aforesaid value is substantially equal to +45° and −45°, according to embodiments of the present invention.

The aforesaid mechanical properties for scaffold 1 may include tensile strength, stiffness, compliance, burst strength and/or suture pull-out force, according to embodiments of the present invention.

In addition to being different from 0° and 180°, angle α1 is different from 90° in order to avoid the weft fibers 24 of weave 20 running parallel to the warp fibers 12 of weave 10, according to embodiments of the present invention.

In order to maintain its mechanical properties in use, scaffold 1 is adapted to secure the weaves 10 and 20 with respect to each other, according to embodiments of the present invention. In other words, in the configuration shown in FIG. 2, the weaves 10 and 20 are bound to each other. In practice, different possibilities can be considered for such a binding.

A first solution is to laminate together the weaves 10 and 20 by interlacing these weaves on a loom: more precisely, after having woven one of the weaves 10 and 20, the other weave is woven on the first one, so that, due to the loom, the weft of this other weave is interlaced with the first one.

Another solution is to laminate together the weaves 10 and 20 by a plurality of stitches. For example, after having woven the two weaves independently from each other, an embroidery machine or a sewing machine may be used to sew stitches across the two weaves in order to bind the weaves together. These stitches may be sewn according to different stitching patterns selected, for example, from the group consisting of straight lines, “E” shapes, “W” or “zigzag” shapes, and the like. As such, mechanical isotropy of scaffold 1 is improved and stabilized by orienting these stitches transversally both to direction D10 and to direction D20.

Another feasible possibility for binding the weaves 10 and 20 may be achieved by interweaving these two weaves during their simultaneous manufacture. Such an interweaving may be performed on a multi-axis loom, although such looms are rare and complex.

In addition to the textile techniques for binding weaves 10 and 20 as discussed above, other techniques can be considered. For example, with an appropriate material constituting the fibers of the weaves 10 and 20, these two weaves can be ultrasonically bonded.

Furthermore, when the weaves 10 and 20 are secured to each other, these two weaves may be advantageously pressed against each other to increase the stability of their binding, to reduce the thickness of scaffold 1 and to improve the compliance of the scaffold.

According to some embodiments of the present invention, scaffold 1 is coated, which increases the mechanical stability of scaffold 1. According to embodiments of the present invention, the material used for such an external coating is selected from the group consisting of collagen, fibrin or other proteins, carboxymethylcellulose, cellulose, chitosan, or other polysaccharides, hyaluronic acid, heparin, heparin sulphate, chondroitin sulphate, or other proteoglycans, and polymers.

In one embodiment, the aforesaid coating forms a porous sponge, especially after having lyophilized an appropriate material dispersion, such as a collagen dispersion. In that way, the external coating does not prevent biologic flows across scaffold 1. Such flows may be desired for the scaffold in view of its use for repairing human tissues, as explained above.

Furthermore, in this context, scaffold 1 may advantageously include biologic agents: these agents may be, for example, impregnated in the weaves 10 and 20, for being released in tissues after implantation of scaffold. Such biologic agents may be selected from the group consisting of growth factors, peptides, proteins, cells, cell extracts, platelet rich plasma, serum, serum extracts, bone marrow extracts, genes, DNA, RNA, siRNA, transcription factors, binding molecules, proteoglycans, carbohydrates, and/or chemokines, according to embodiments of the present invention.

According to one embodiment, the fibers 12, 14, 22 and 24 of the weaves 10 and 20 are made of polyhydroxyalkanoate, especially of TephaFlex (registered mark) which corresponds to poly-4-hydroxybutyrate.

Based on the disclosure provided herein, one of ordinary skill in the art will appreciate that other biocompatible materials, especially resorbable polymers, may be used.

In practice, each of the fibers 12, 14, 22, 24 may be a monofilament: Monofilaments maintain space or pores between their intersections which encourages bone and soft tissue growth into scaffold 1. In addition or in substitution of such monofilaments, multifilaments may be used for forming the aforesaid fibers; in that way, the binding capacity of the weaves 10 and 20 is increased and can be sufficient even without an applied coating. When a multiplicity of such multifilaments are used, the external surfaces of scaffold 1 achieve a felt effect, which can be useful for specific implantations, according to embodiments of the present invention.

Leno weave 10 shown in FIG. 3 is not the only one woven structure that can be considered for each leno weave of scaffold 1. FIG. 4 depicts a variant for weave 10, which is referenced 10′. Contrary to weave 10, in which only one weft fiber 14 is systematically interposed along direction D10, between two successive intersections of the two-warp fibers 12 of each of pairs 15, two successive intersections 16′ between the two warp fibers 12′ of pairs 15′ constituting warp 11′ of weave 10′ are alternatively separated by one of the fibers 14′ of weft 13′ and by two of these weft fibers. In this way, the packing of the fibers in weave 10′ may be increased to increase density, according to embodiments of the present invention.

As shown by FIG. 4, for each of the pairs 15′, two successive intersections 16′, which are separated by two weft fibers 14′, are positioned along direction D10′ of weave 10′, in a shifted manner with respect to the two adjacent pairs 15′. In other words, the intersections 16′ (where the warp fibers 12′ cross) alternate in direction D10′, which permits denser packing of the warp pairs 15′, according to embodiments of the present invention.

FIG. 5 depicts another variant for weave 10, which is referenced 10″, according to embodiments of the present invention. Compared to weave 10′, two weft fibers 14″ of weft 13″ are systematically interposed between two successive intersections 16″ between the two warp fibers 12″ of each of the warp pairs 15″ constituting warp 11″ of leno weave 10″. In this way, the packing of the weft fibers 14″ is permitted to be denser.

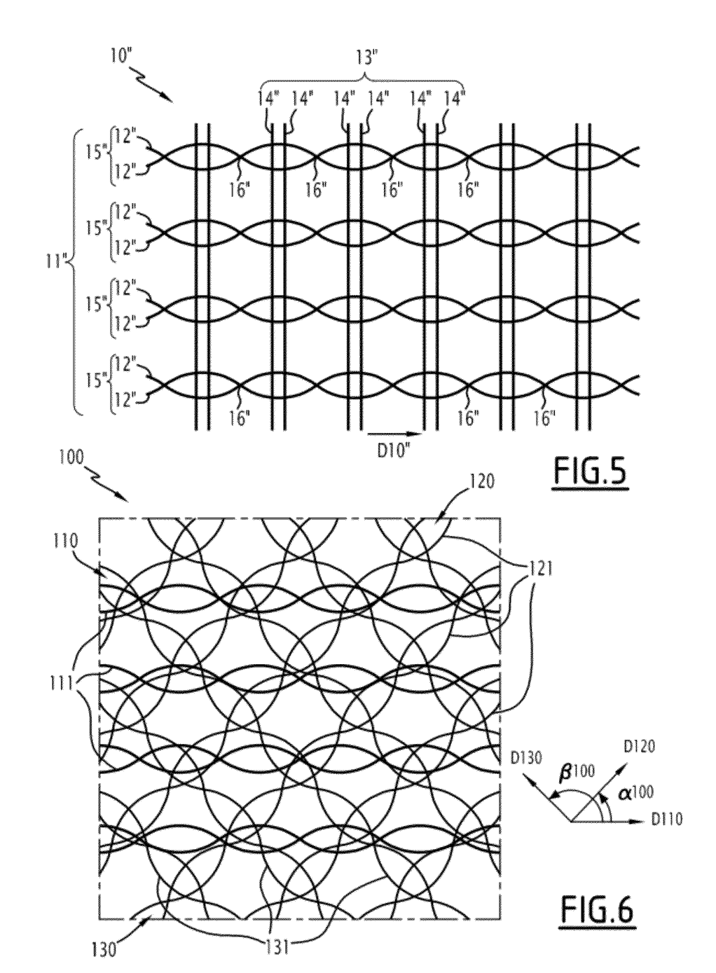

FIG. 6 depicts a scaffold 100, which essentially differs from scaffold 1 by the number of its weaves: scaffold 100 comprises three weaves 110, 120 and 130, according to embodiments of the present invention. Taken individually, these weaves 110, 120 and 130 may be identical and may each have the same structure of weave 10 or 20. In particular, each of these weaves 110, 120 and 130 includes both a warp 111, 121, 131, oriented in its respective direction D110, D120, D130, and a weft, not drawn in FIG. 6 for clarity reasons, oriented perpendicularly to the aforesaid direction D110, D120, D130.

The three weaves 110, 120 and 130 are superimposed in a parallel manner and are bound to each other so that any one of the directions D110, D120 and D130 is non-parallel to the others, according to embodiments of the present invention. In other words, as indicated in FIG. 6, an angle α100 is formed between the directions D110 and D120 of the weaves 110 and 120, this angle α100 being different from 0° and 180°. And an angle β100 is formed between the directions D110 and D130 of the weaves 110 and 130, this angle β100 being different from 0°, 180° and α100.

According to a preferred embodiment, the angles between the three directions D110, D120 and D130 are regularly or uniformly distributed in the plane of parallelism between the weaves 110, 120 and 130 of scaffold 100. In other words, in an angularly oriented plane, angle α100 has a value of about +60° and angle β100 has a value of about +120°, according to embodiments of the present invention.

Suitable structures can also be made by interspersing small zones of leno weave with plain weave to increase the density and strength of the scaffold, by laminating leno woven layers with plain weave layers, or by weaving monofilaments in a plain weave intermixed with multifilament yarns woven in a leno manner to give higher strength, maintain porosity, and still produce stable scaffolds that can be trimmed to shape, according to embodiments of the present invention.

In practice, each of the various arrangements and variants which have been described for scaffold 1 may be implemented for scaffold 100.

According to some embodiments of the invention, the scaffolds such as described here above may be placed between a bone and a soft tissue, or between two bones surfaces, or between two soft tissues, in a human body or in an animal. In that way, bone ingrowth and/or soft tissue ingrowth are promoted. In the interface between the two tissues, which does not collapse and is not crushed or displaced, Sharpey's fibers may be formed and/or a volume may be stabilized for new callus formation. In practice, the porous scaffold which is used presents:

a stiffness corresponding to 10 to 90 % of the stiffness of the tissue to be replaced, and/or

a porosity of at least 10% under a compressive load of 25 psi, and/or

the pores thereof being larger than 50 micrometers in diameter.

According to some embodiments of the invention, the scaffolds such as described herein may provide a transition, for example a mechanical transition, between a bone tissue and a soft tissue, instead of abrupt change in modulus. In other words, the scaffolds provide a gradient, for example a gradient in stiffness, in the interface between the two tissues.

As explained above, the various embodiments according to embodiments of the invention include the textile types, the textile openness (porosity), the textile thickness, the textile density, the used materials, at least one potential treatment, especially coating, to these materials that changes their properties, and/or the combinations of the used textile with potential added agents

According to embodiments of the present invention, different surgical methods may used for placing the scaffolds such as those described herein. For example, such a scaffold may be inserted between the two tissues. Or the scaffold may be anchored to one of the two tissues before reattaching the other one to the first one in a specific region.

### EXAMPLE 1

A sample of the textile structure described in FIG. 2 woven with 150 um diameter TephaFlex (registered mark) monofilament in a leno pattern with the angle between plies of 45 degrees and sewn together with the same TephaFlex (registered mark) monofilament fiber was tested wet on a mechanical testing machine. For the tensile strength a 2 cm by 7 cm strip was mounted in wedge grips with a gauge length of 2.5 cm, cycled 30 times between 22 and 133 N, and then pulled to failure at 1 KN/min. For the suture pull-out testing a 2 cm by 3 cm strip of the weave was mounted in a wedge grip leaving a gauge length of 2.5 cm. A size 2 ForceFiber high strength suture was tied in a 5 mm wide mattress stitch placed 5 mm from the opposite edge of the strip. The suture was then gripped and pulled at 1 KN/min until failure. Table 1, below, reflects these results in a comparative table.

TABLE 1

| | Ultimate Stress | Tensile Stiffness | Suture Pull- |
|---|---|---|---|
| Product | MPa | N/mm | out N |
| supraspinatus tendon | 38* | 97* | |
| Example 1 | 33 | 68 | 180 |
| OrthAdapt | | | 44† |
| Restore | 10* | 7* | 38† |
| ZCR | | | 128† |
| GraftJacket | 7* | 16* | 157† |

*A Aurora, et al, JSES, 16 (5S), 2007, pp 171S-178S.
†F A Barber, et al, Arthroscopy 22(5), 2006, pp 534-538.

### EXAMPLE 2

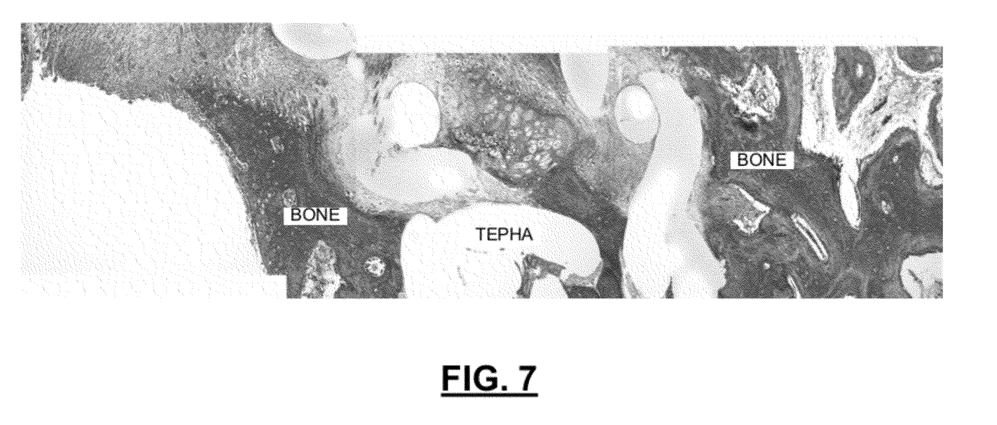

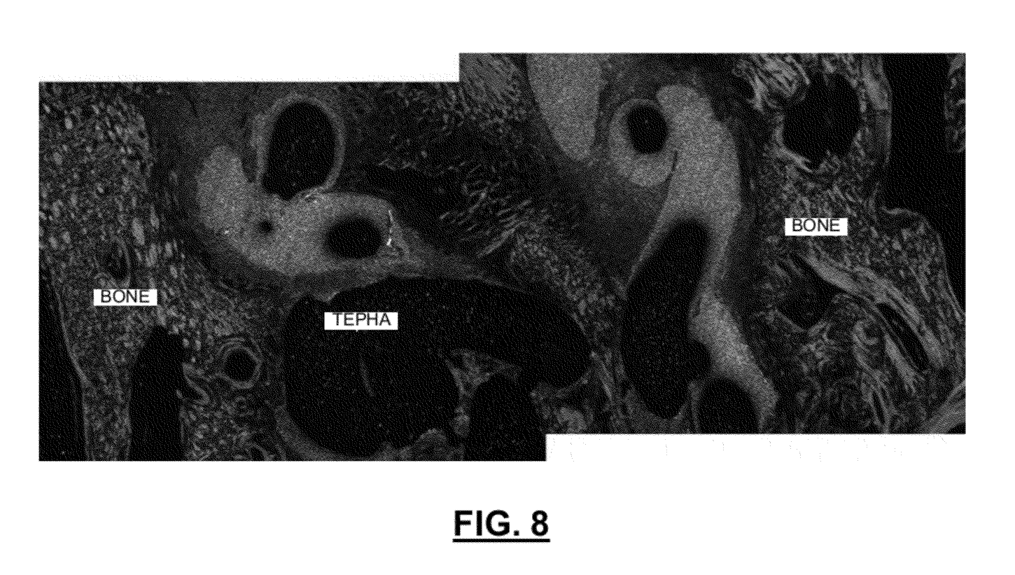

The woven scaffold described in Example 1 was used to repair the infraspinatus tendon in a mature sheep. Three months after the surgery the shoulder was examined histologically. Where the scaffold was in contact with tendon tissue, the tissue ingrew throughout the scaffold. Where the scaffold was in contact with bone on the humerus bone was observed to grow right through the scaffold until it was encased. A new interface between bone and tendon was observed within and adjacent to the scaffold which included the Sharpey's fibers found in normal bone/tendon interfaces. This ingrowth is illustrated in the histological photographs of FIGS. 7 and 8.

More generally, other embodiments of the invention are possible, including by recombining the various elements disclosed herein in different or alternative combinations. Although the above description contains many specifics, these should not be considered as limiting the scope of the invention as defined by the appended claims, but as merely providing illustrations of some of the embodiments of this invention.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A method for implanting an endoprosthetic textile scaffold, comprising:
inserting a bi-layer woven endoprosthetic textile scaffold between bone and soft tissue, the bi-layer woven endoprosthetic textile scaffold promoting healing between the bone and the soft tissue; and
anchoring the soft tissue to the bone.

2. The method of claim 1, further comprising cutting the bi-layer woven endoprosthetic textile scaffold to a desired shape.

3. The method of claim 1, wherein the soft tissue is shoulder rotator cuff tissue.

4. The method of claim 1, wherein the bi-layer woven endoprosthetic textile scaffold comprises a first layer and a second layer joined together, the first and second layers being substantially parallel, wherein a warp element of the first layer is non-parallel to a warp element of the second layer.

5. The method of claim 4, wherein the first and second layers are each formed with a leno weave.

6. An endoprosthetic textile scaffold comprising:
a first woven layer comprising a first warp element oriented substantially along a first direction and a first weft element oriented substantially perpendicularly to the first direction;
a second woven layer comprising a second warp element oriented substantially along a second direction and a second weft element oriented substantially perpendicularly to the second direction;
wherein the first woven layer is attached to the second woven layer in a parallel manner, and wherein the first direction is non-parallel to the second direction.

7. The endoprosthetic textile scaffold of claim 6, wherein an angle between the first direction and the second direction is not zero, ninety, or one hundred eighty degrees.

8. The endoprosthetic textile scaffold of claim 7, wherein the angle is substantially forty-five degrees.

9. The endoprosthetic textile scaffold of claim 6, further comprising:
a third woven layer comprising a third warp element oriented substantially along a third direction and a third weft element oriented substantially perpendicularly to the third direction;
wherein the first, second, and third woven layers are attached together in a parallel manner, and wherein the third direction is non-parallel to the first direction and to the second direction.

10. The endoprosthetic textile scaffold of claim 9, wherein a first angle formed between the first and second directions is substantially sixty degrees, and wherein a second angle formed between the first and third directions is substantially one hundred twenty degrees.

11. The endoprosthetic textile scaffold of claim 6, wherein the first woven layer and the second woven layer are each formed with a leno weave.

12. The endoprosthetic textile scaffold of claim 6, wherein one or both of the first warp element and the second warp element comprises two or more warp fibers which cross each other once between each weft element.

13. The endoprosthetic textile scaffold of claim 12, wherein one or both of the first weft element and the second weft element comprises two or more weft fibers.

14. The endoprosthetic textile scaffold of claim 6, wherein one or both of the first weft element and the second weft element comprises two or more weft fibers.

15. The endoprosthetic textile scaffold of claim 6, wherein one or both of the first warp element and the second warp element comprises two or more warp fibers which cross each other once between every second weft element.

16. The endoprosthetic textile scaffold of claim 15, wherein one or both of the first weft element and the second weft element comprises two or more weft fibers.

17. The endoprosthetic textile scaffold of claim 6, wherein one or more of the first warp element, the second warp element, the first weft element, and the second weft element comprises a monofilament fiber.

18. The endoprosthetic textile scaffold of claim 6, wherein one or more of the first warp element, the second warp element, the first weft element, and the second weft element comprises a multifilament fiber.

19. The endoprosthetic textile scaffold of claim 6, wherein one or more of the first warp element, the second warp element, the first weft element, and the second weft element is made of polyhydroxyalkanoate.

20. The endoprosthetic textile scaffold of claim 6, wherein the first woven layer is attached to the second woven layer by interlacing the weft of the second woven layer with the first woven layer.

21. The endoprosthetic textile scaffold of claim 6, wherein the first woven layer is attached to the second woven layer by a stitching pattern, wherein at least portions of the stitching pattern are oriented transversely to both the first and second directions.

22. The endoprosthetic textile scaffold of claim 6, wherein the first woven layer is attached to the second woven layer by interweaving the first woven layer with the second woven layer during simultaneous manufacture of the first and second woven layers.

23. The endoprosthetic textile scaffold of claim 6, wherein the first woven layer is ultrasonically bonded to the second woven layer.

24. The endoprosthetic textile scaffold of claim 6, further comprising a coating forming a porous sponge.

25. The endoprosthetic textile scaffold of claim 6, wherein the first woven layer and the second woven layer are made of biocompatible materials for implantation in the body.

26. The endoprosthetic textile scaffold of claim 6, wherein the first woven layer and the second woven layer are made of bioresorbable monofilaments having a diameter larger than fifty micrometers.
